(19) United States
(12) Patent Application Publication
(10) Pub. No.: US 2017/0296081 A1
(43) Pub. Date: Oct. 19, 2017

(54) FRAME BASED SPIKE DETECTION MODULE

(71) Applicant: Agency for Science, Technology and Research, Singapore (SG)

(72) Inventors: Lei Liao, Singapore (SG); Xin Liu, Singapore (SG)

(21) Appl. No.: 15/518,128

(22) PCT Filed: Oct. 16, 2015
(86) PCT No.: PCT/SG2015/050395

§ 371 Date: Apr. 10, 2017

(30) Foreign Application Priority Data
Oct. 16, 2014 (SG) 10201406687V

(51) Int. Cl. A61B 5/04; A61B 5/0476; A61B 5/00; A61B 5/0205; A61B 5/0432

(52) U.S. Cl. CPC A61B 5/04012; A61B 5/04001; A61B 5/0476; A61B 5/0432; A61B 5/0006; A61B 5/0205; A61B 5/0031; A61B 2560/0475

(57) ABSTRACT

A method and device for biomedical recording of biomedical spike signals is provided. The method includes extracting and aligning possible biomedical spike signals from received signals and, thereafter, performing spike detection by determining whether the possible biomedical spike signals are actual spike signals. The biomedical spike signals are preferably selected from electrocardiography (ECG), electroencephalography (EEG) and neural signals.

### PRIORITY CLAIM

This application claims priority from Singapore Patent Application No. 10201406687V filed on Oct. 16, 2014.

### TECHNICAL FIELD

The present invention generally relates to methods and apparatus for biomedical signal recording, and more particularly relates to methods and apparatus for providing frame based biomedical signal spike detection.

### BACKGROUND OF THE DISCLOSURE

Biomedical signal recording systems are a tool for doctors and scientists to view biomedical operations. Biomedical signals can include electrocardiography (ECG), electroencephalography (EEG) and neural signals. As one example, implantable neural recording systems are important brain-machine interfaces which make neuron activity accessible to neuroscientists. Extracellular data recording and wireless data transmission are two essential steps for such recording systems. A high sampling frequency ranging between 20 kHz and 30 kHz is typically required in order to capture the large bandwidth neural signals. As a result, a substantial amount of recorded data will be generated for wireless transmission. Due to the vulnerability of body tissues, however, there are critical requirements on size, heat emission and power consumption of such implantable neural recording systems. Thus, due to the limitation of communication bandwidth in brain-machine interfaces as well as the constraints on power consumption, on-chip data reduction becomes a necessary task to perform before wireless transmission.

Traditional setups for neural recording require high bandwidth communications between recording electrodes and the processing computer because the spikes are detected and sorted at the computer. When more recording electrodes are deployed, transmission resources become insufficient and energy-hungry. Therefore, on-chip spike detection and transmission is desirable to reduce the wireless transmission load. However, existing on-chip spike detectors do not have alignment capability and, thus, additional hardware is needed for alignment. Furthermore, detection independent of alignment increases the missed detection rate. Existing on-chip detectors are designed either based on PC-level detection methods which are complex and energy consuming, or based on simple magnitude thresholding which is prone to detection error.

Since all neuron actives are represented by the potential firing processes which follows an on-off spike pattern, on-chip spike detection enables data reduction since only detected spikes are transmitted. As a result, the transmission load will be reduced and significant power saving for wireless transmission can be achieved.

Existing on-chip spike detector modules can be broadly classified into two categories: non-blind detection modules and blind detection modules. The main difference between these two categories is the presence of a template of the target spike. Non-blind spike detectors attempt to identify the event from among raw data that closely matches a given spike template. These detectors are typically based on the methods of sum-of-squared differences, maximum likelihood estimation and matched filter. The non-blind spike detector is less preferred as it is impractical in implementations of non-blind spike detectors to obtain accurate information of target spikes.

Among the proposed blind spike detectors, those basing their detection on amplitude thresholding achieves the most significant hardware cost saving as it only searches for an event that crosses a predefined threshold from selected raw data. Both user-specified and automatically-evaluated amplitude thresholding rules can be adopted. A training session is required in order to estimate a standard deviation of noise so that the threshold can be estimated as a multiple of its medium value. It has been further reported that better results can be achieved by applying a threshold to the absolute value of the data. Effectively, this applies double-sided thresholds to the recorded data since spikes can be either positive or negative. Although these methods are computationally efficient and easy to be implemented in hardware, the main drawback is performance degradation as the noise level increases. Instead of measuring the amplitude of the recorded signal directly, the non-linear energy operator (NEO) evaluates the energy difference between neighboring samples. Since NEO takes into account two neighborhood data points (one ahead and one behind) in addition to the current input data, the NEO often outperforms the direct thresholding method as it explores the time-frequency information of the raw data.

Another important group of methods utilizing time-frequency analysis is discrete wavelet transform (DWT) based approaches. The advantage of DWT-based on-chip spike detectors is their hardware cost efficiency of decomposing the time-domain recorded data into a time-frequency domain. DWT-based detectors can provide good time resolution and relatively poor frequency resolution at high frequencies, while good frequency resolution and poor time resolution at low frequencies. This characteristic is useful because most natural biomedical signals, such as ECG, EEG and neural signals, have low frequency content spread over long duration and high frequency content for short durations. The success of DWT-based approaches depend on the choice of wavelet and the number of decomposition levels. However, both amplitude thresholding and DWT-based detectors are unable to achieve both high accuracy and robustness against noise and DC drifting simultaneously

Thus, what is needed is a method and device for biomedical signal spike detection which at least partially overcomes the drawbacks of present approaches. Furthermore, other desirable features and characteristics will become apparent from the subsequent detailed description and the appended claims, taken in conjunction with the accompanying drawings and this background of the disclosure.

### SUMMARY

According to at least one embodiment of the present invention a method for method for biomedical signal recording is provided. The method includes extracting and aligning possible biomedical spike signals from received signals and, thereafter, performing spike detection by determining whether the possible biomedical spike signals are actual spike signals.

In accordance with another aspect of at least one embodiment of the present invention, a biomedical signal recording device is provided. The biomedical recording device includes a preliminary alignment module and a spike detection module. The preliminary alignment module extracts possible biomedical spike signals from received signals while automatically aligning the possible biomedical spike signals. The spike detection module is coupled to the preliminary alignment module for receiving the possible biomedical spike signals therefrom and determines whether the possible biomedical spike signals are actual spike signals.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with a present invention, by way of non-limiting example only, wherein:

FIG. 1, comprising FIGS. 1A, 1B and 1C, illustrates graphs of various biomedical signals, wherein FIG. 1A depicts a graph of an electroencephalography (EEG) signal, FIG. 1B depicts a graph of a neural signal and FIG. 1C depicts a graph of an electrocardiography (ECG) signal;

FIG. 2 illustrates a block diagram of a neural recording device in accordance with a present embodiment;

FIG. 3 illustrates; a graph of preliminarily aligned neural spikes in accordance with the present embodiment

FIG. 4, comprising FIGS. 4A, 4B and 4C, illustrates graphs of various signals in a first simulation of the neural recording device in accordance with the present embodiment, wherein FIG. 4A depicts a graph of raw data signals received from the analog-to-digital converter (ADC) of the neural recording device in accordance with the present embodiment, FIG. 4B depicts a graph of data signals received from the energy clustering calculator of the neural recording device in accordance with the present embodiment, and FIG. 4C depicts a graph of aligned actual neural spike signals in accordance with the present embodiment;

FIG. 5, comprising FIGS. 5A, 5B and 5C, illustrates graphs of various signals in a second simulation of the neural recording device in accordance with the present embodiment where the target spikes are subject to large DC drifting, wherein FIG. 5A depicts a graph of raw data signals including designed spikes with a single tone noise used for the simulation of the neural recording device in accordance with the present embodiment, FIG. 5B depicts a graph zooming in on a spike of the data signals of FIG. 5A, and FIG. 5C depicts a graph of spike and noise signals of the simulation as received from the energy clustering calculator of the neural recording device in accordance with the present embodiment.

FIG. 6, comprising FIGS. 6A to 6D, illustrates graphs of various signals in a third simulation of the neural recording device in accordance with the present embodiment where the signals include high recording noise, wherein FIG. 6A depicts a graph of raw data signals with a signal-to-noise ratio (SNR) of approximately ten decibels (10 dB) for the simulation of the neural recording device in accordance with the present embodiment, FIG. 6B depicts a graph zooming in on a spike of the data signals of FIG. 6A, FIG. 6C depicts a graph further zooming in on a spike of the data signals of FIG. 6A, and FIG. 6D depicts a graph of spike and noise signals of the simulation as received from the energy clustering calculator of the neural recording device in accordance with the present embodiment; and

FIG. 7 depicts a flowchart of a method for neural recording in accordance with the present embodiment.

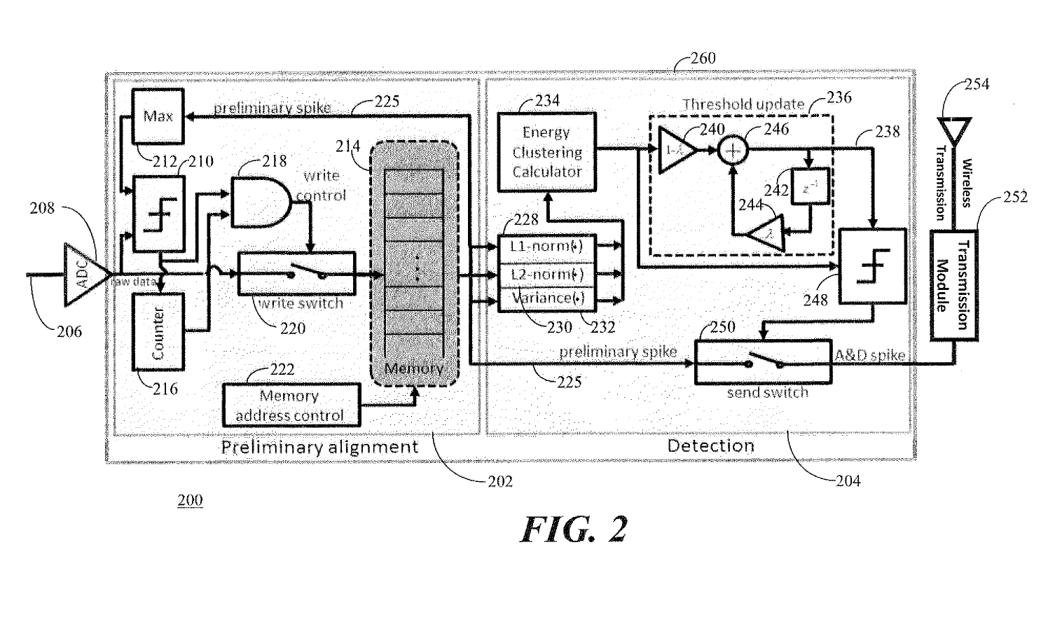

Skilled artisans will appreciate that elements in the figures are illustrated for simplicity and clarity and have not necessarily been depicted to scale. For example, the elements of the block diagram of FIG. 2 have been enlarged for easy viewing and the blocks may not be accurate in respect to size of other blocks of the neural recording device in accordance with the present embodiment.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. It is the intent of this invention to present a system and method for highly accurate real-time spike detection that is suitable for biomedical devices. The system has low complexity and low energy consumption while simultaneously extracting and aligning possible spike signals and thereafter detecting whether the possible spike signals are actual spike signals.

Biomedical signals such as electrocardiography (ECG) and electroencephalography (EEG) include potentially firing spike-like signals, like neural signals, whose energy are highly centralized (i.e., a small number of samples dominate the whole frame). The following terms are defined in accordance with a present embodiment. A “spike” is a peak signal (e.g., a potentially firing neural spike-like signal). A “spike signal” is defined as a portion of data which contains a peak (i.e., a spike) as well as some samples before and after the peak. A “frame” is defined as the portion of data of the spike signal which has a predefined data width.

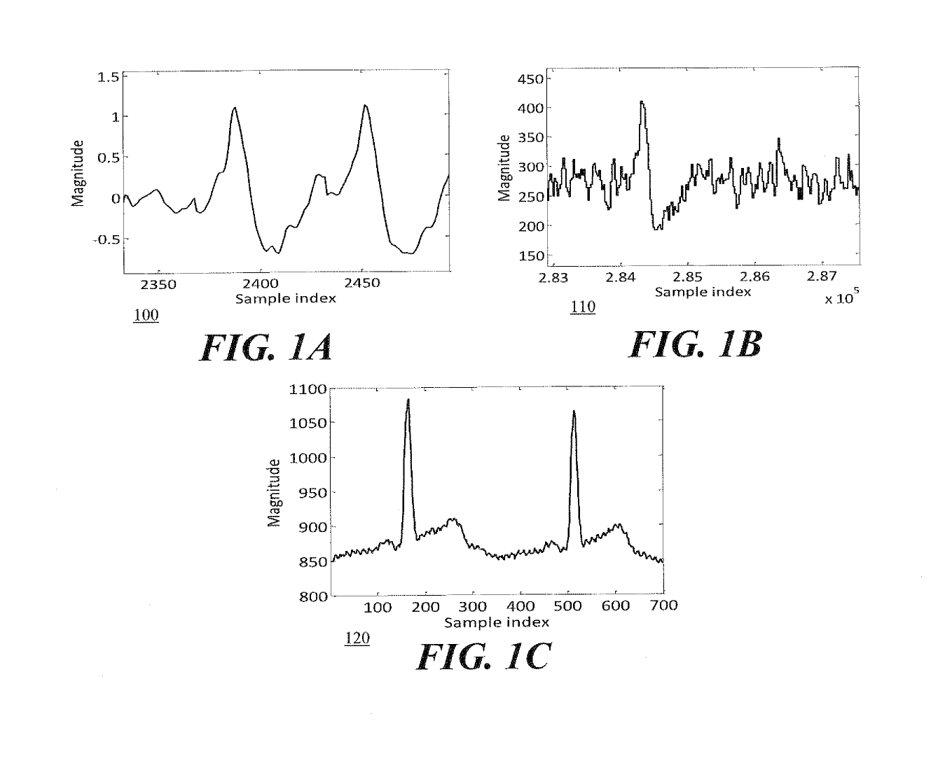

“Energy clustering” is defined as clustering of the amplitude of signals within a data frame. Spike signals and noise signals are distinct in terms of the degree of energy clustering of the signal. Referring to FIG. 1, it can be seen that spikes obtained from various biomedical activities share a common feature. FIG. 1A illustrates a graph 100 of an EEG signal, FIG. 1B illustrates a graph 110 of a neural signal and FIG. 1C illustrates a graph of an ECG signal. The shared common feature of these biomedical signals is that the spikes obtained from various biomedical activities cluster their energy in a small region. Thus, evaluation of a preliminary spike using energy clustering in accordance with the present embodiment is realized by utilizing this shared common feature of biomedical signals.

“Frame-based energy clustering” is defined as energy clustering of the signals within a data frame. Frame-based energy clustering is only sensitive to the relative difference among the samples while it is not sensitive to baseline drifting. And an “accelerator” is defined as a calculator which performs a predetermined function with a data frame to enhance contrast of multiple data samples of the data frame in order to enhance frame-based energy clustering calculations of the data frame.

Existing detector modules are sensitive to noise signals which are dispersive and generally randomly distributed (i.e., the energy of noise signals typically tends to be equally distributed). In accordance with the present embodiment, preliminary spike alignment is performed as a first step of data reduction since only those frames satisfying the specified spike alignment criteria are sent for energy clustering calculations, thereby reducing the energy clustering calculations to provide reduced energy consumption without reducing the accuracy of spike detection. Accuracy is maintained because the preliminary spike alignment ensures the all potential spikes are measured using energy clustering calculator thereby reducing the miss detection rate. In addition, further energy consumption reduction can be obtained and hardware cost and size can be reduced by reusing the energy clustering index of aligned spikes in the possible spike sorting.

Conventional spike detectors perform spike detection and spike alignment independently without considering the local maxima problem. This leads to false alarming as the peak identified might not be the maxima across the entire frame. In order to address this problem and save hardware cost on spike alignment, a novel hardware architecture in accordance with the present embodiment aligns the preliminary spike first and subsequently performs spike detection.

Referring to FIG. 2, a block diagram 200 depicts a neural recording device in accordance with the present embodiment. The neural recording device includes a preliminary alignment module 202 and a spike detection module 204. A front-end signal 206 undergoes conversion by an analog-to-digital converter (ADC) 208 to become discrete-time digital data. Each incoming data is compared by a comparator 210 with a maximum value 212 of data stored in memory cells of a memory 214 whose size is equal to the predefined data width of the frame (i.e., the data width of a spike signal). The output of the comparator 210 is connected to a counter 216 for controlling an alignment position and a spike length of data to be stored in the memory 214, both of which can be pre-adjusted for different applications. The output of the comparator 210 is also connected to a write control device 218 which utilizes the comparison of the incoming data from the ADC 208 and the maximum value 212 to determine if the new data will be stored into the memory 214 and to activate a write switch 220 to store the new data into the memory 214, a memory cell address of the data in the memory 214 being assigned by a memory address control unit 222. A preliminary spike signal 225 is obtained automatically once all memory cells in the memory 214 are full.

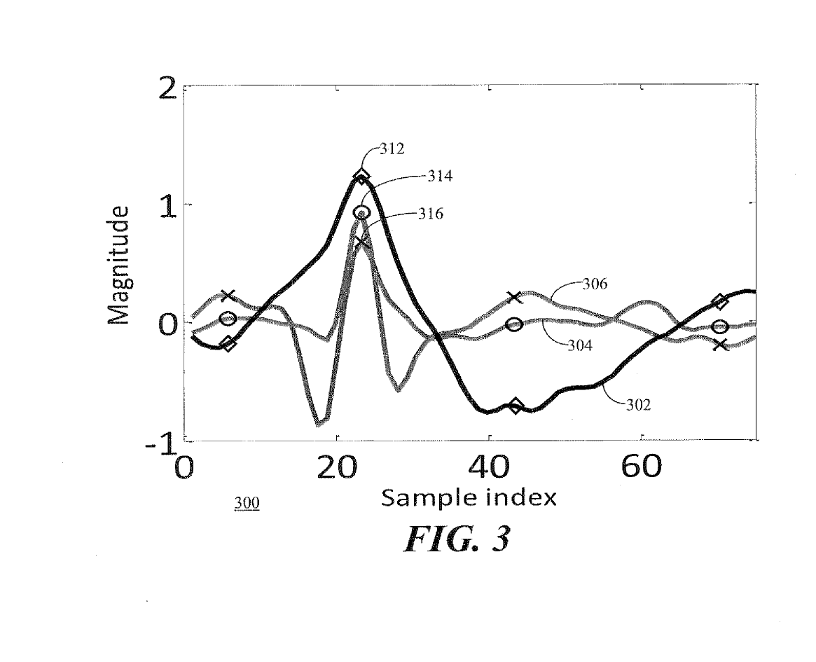

Referring to FIG. 3, a graph 300 depicts frames of preliminarily aligned neural spike signals 302, 304, 306 in accordance with the present embodiment where the respective peaks 312, 314, 316 are aligned. The spike signal peaks 312, 314, 316 are preliminarily aligned at a user specified position by aligning the data in the memory cells of the memory 214 in response to memory cell addressing by the memory address control 222.

Referring back to FIG. 2, the preliminary spike signal 225 is passed not only to the maximum value 212, but also to a plurality of accelerators 228, 230, 232 and to a send switch 250. The plurality of accelerators 228, 230, 232 a L1-norm accelerator 228, a L2-norm accelerator 230 and a variance accelerator 232. The L1-norm accelerator 228 calculates the sum of the absolute value of all the data within the preliminary spike signal. The L2-norm accelerator 230 calculates the squared root of the sum of squared values of each data in the preliminary spike signal. And the variance accelerator 232 determines a variance of the data in the preliminary spike signal. The results from the plurality of accelerators 228, 230, 232 is used by an energy clustering (EC) calculator 234 which performs the following calculation

$$EC ( {x}_{L} ) = a \times var ( {x}_{L} ) + ( 1 - a ) \frac{{‖ {x}_{L} ‖}_{2}}{{‖ {x}_{L} ‖}_{2} + {eps}^{′}} \qquad ( 1 )$$

where x_{L} denotes the preliminary spike signal, var{•} denotes the variance computation by the variance accelerator 232, eps is a sufficiently small constant that prevents zero division, and a is a weighting factor that can either be a constant or a time-varying variable. It should be noted that the energy clustering calculator 234 extracts a single valued feature from the multiple data samples of the preliminary spike signal. In accordance with the present embodiment, the preliminary spike is determined to be highly energy clustered if EC{x_{L}} is large.

Frame-based energy clustering in accordance with the present embodiment performs advantageous spike detection by energy clustering after quantitatively measuring the relative difference between all the data within the frame by the variance accelerator 232 and ensuring the sensitivity of the energy clustering measurement to the variation within the discrete-time series by energy clustering after the L1-norm accelerator 228 and the L2-norm accelerator have enlarged the contrast between the data in the frame by the ratio between the L1-norm calculation and the L2-norm calculation.

A dynamic threshold module 236 is coupled to the energy clustering calculator 234 to define a clear threshold line to extract spike data by a dynamic spike threshold comparison by a comparator 248 with the energy clustered possible spike signal from the energy clustering calculator 234 to determine whether the energy clustered possible spike signal is an actual spike signal. The dynamic threshold module 236 generates a new threshold 238 which is derived from its previous value and the current output from the energy clustering calculator 234 for each possible spike in the following manner: the current output from the energy clustering calculator 234 is multiplied by forgetting factor λ 240 which has a range from zero to one and is normally close one; the result is delayed by z⁻¹ 242, where z⁻¹ is the standard delay unit in digital signal processing and then multiplied by the forgetting factor λ 244; then the sum 246 of the delayed signal (i.e., the previous output from the energy clustering calculator 234) and the present output from the energy clustering calculator 234 generates the dynamic threshold value 238. The larger the λ, the higher weight is given to previous information. When λ is set to one (1), a static threshold is effectively adopted. In this manner, the dynamic threshold module 236 dynamically updates the spike threshold in response to the extracted single valued feature from the energy clustering calculator 234 and the comparator 248 compares the extracted single valued feature with the dynamically updated spike threshold to determine whether the energy clustered possible spike signals are actual spike signals.

When the comparison by the comparator 248 is positive (i.e., the possible spike signal is determined to be an actual spike signal) the send switch 250 is closed and the spike signal is encoded by a transmission module 252 for wireless transmission from an antenna 254 in a manner well known to those skilled in the art. Thus, transmission circuitry necessary for wirelessly transmitting the actual biomedical signals includes the transmission switch 250, the transmission module 252 and the antenna 254 and the transmission switch 250 operates under control of the spike detection module 204 for forwarding the actual biomedical spike signals to the transmission module 252 for wireless transmission. For implantable wireless neural recording of neural spike signals, a biocompatible housing 260 encloses the preliminary alignment module 202 and the spike detection module 204 to permit internal implantation in a subject.

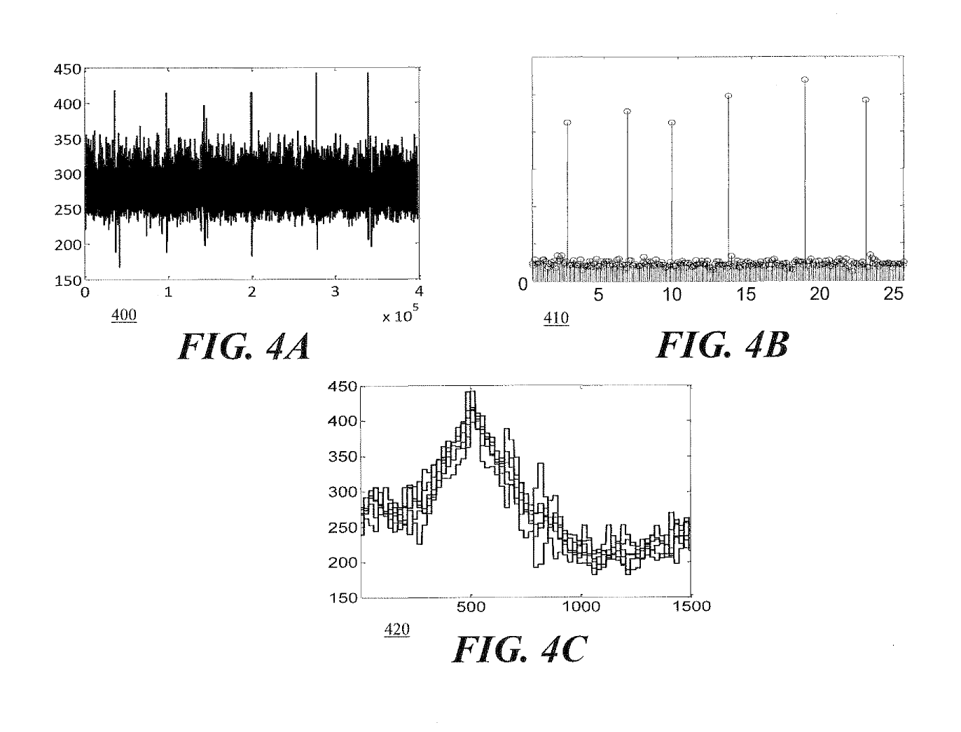

Referring next to FIG. 4, FIG. 4A depicts a graph 400 of discrete-time digital data signals received from the ADC 208 containing six neural spikes in accordance with the present embodiment. The data signals were recorded from test rats using a frequency of 12.5 kHz with a resolution of nine bits. FIG. 4B depicts a graph 410 of data signals received from the energy clustering calculator 234 measuring all the preliminary spike signals. FIG. 4C depicts a graph 420 of aligned neural spike signals as provided as the preliminary spike signal to the send switch 250.

It can be observed from the graph 400 that the target six spikes are surrounded by high recording noise. Applying direct magnitude thresholding is prone to inaccurate detection as such thresholding is not able to draw a clear threshold line to differentiate the spikes and noise. In contrast, as can be seen from the graph 410, the energy clustering measure of all preliminary spikes by the proposed spike detector exhibits significant difference. In total, there are 266 preliminary spikes detected. It can be observed from the graph 410 that the target six spikes have a much larger energy clustering than the other preliminary spikes, implying that those preliminary spikes with significant higher energy clustering is a real spike while the others are noise. In addition, a clear threshold line can be determined to extract the spikes. The graph 420 presents the final spikes detected by the proposed module. It can be observed that all six spikes are successfully detected and aligned automatically. As a result, only these six spikes are sent from the send switch 250 for transmission, thus achieving a total energy saving in wireless transmission of 266−6/266=97.74% by operation in accordance with the present embodiment.

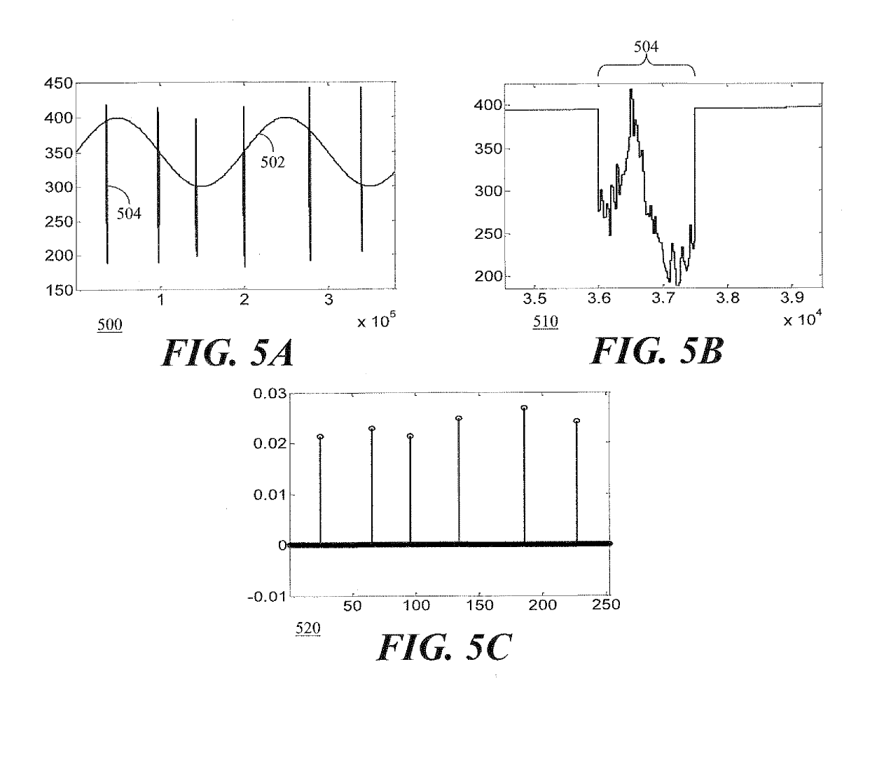

While FIG. 4 involved detection of spikes in a high noise environment, FIG. 5 depicts simulation results of detection in accordance with the present embodiment where the target spikes are subjected to large DC drifting during recording. FIG. 5A depicts a graph 500 of raw data signals including designed spikes with a single tone noise 502 used for the simulation. FIG. 5B depicts a graph 510 zooming in on a spike 504 of graph 500. It can be observed from the graphs 500, 510 that the target spikes sit on a sinusoidal DC drift. The magnitude thresholding rule will completely fail as the drifting signal has comparable magnitude with the spike signals so that no thresholding line can be drawn. However, as can be observed in FIG. 5, a graph 520 of spike and noise signals of the simulation as received from the energy clustering calculator 234 indicates that spike alignment and detection in accordance with the present embodiment still differentiates the target spikes from drifting signal successfully.

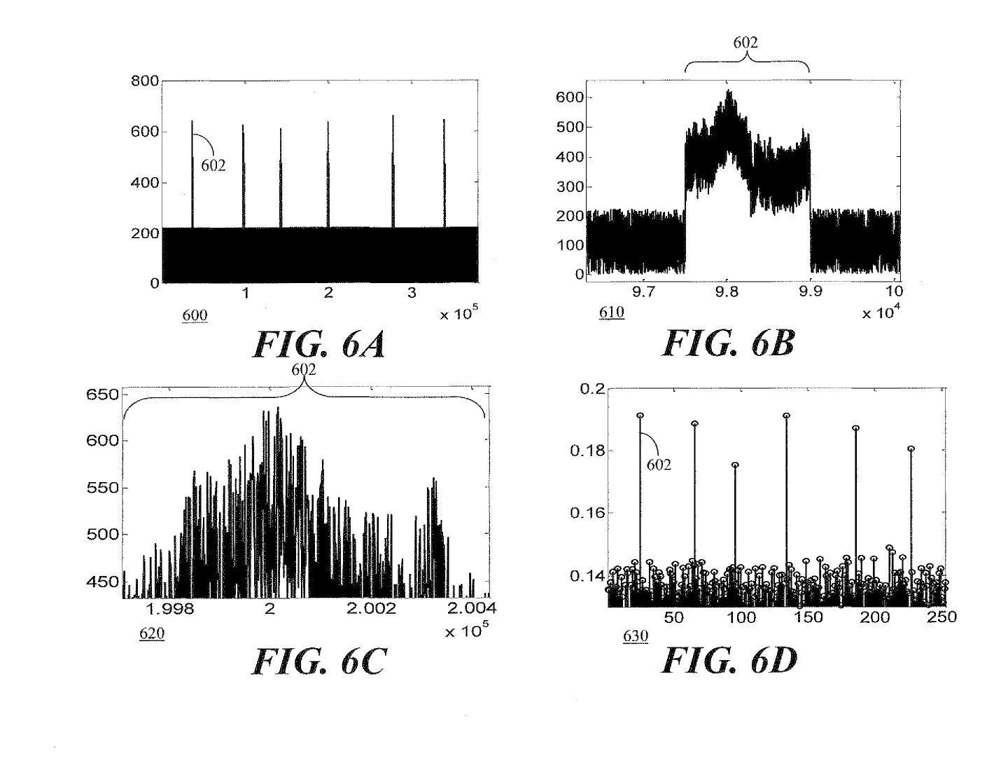

Referring to FIG. 6, a further simulation of operation of a neural recording device in accordance with the present embodiment where high recording noise is present. FIG. 6A depicts a graph 600 of raw data signals with a signal-to-noise ratio (SNR) of approximately ten decibels (10 dB). FIG. 6B depicts a graph 610 zooming in on a spike signal 602 of the data signals of the graph 600 and FIG. 6C depicts a graph 420 further zooming in on the spike signal 602. It can be observed from the zoomed-in recorded signal 602 in graphs 610 and 620 that the noise is high with multiple local maxima that can be identified by magnitude thresholding. This type of spike signal can lead to false alarming and low detection accuracy in conventional neural recording devices. However, as can be seen from FIG. 6D which depicts a graph 430 of spike and noise signals of this simulation as received from the energy clustering calculator 234 of the neural recording device in accordance with the present embodiment, the present embodiment overcomes the drawbacks of conventional methods and provides increased accuracy in spike detection, including accurate detection of the spike signal 602.

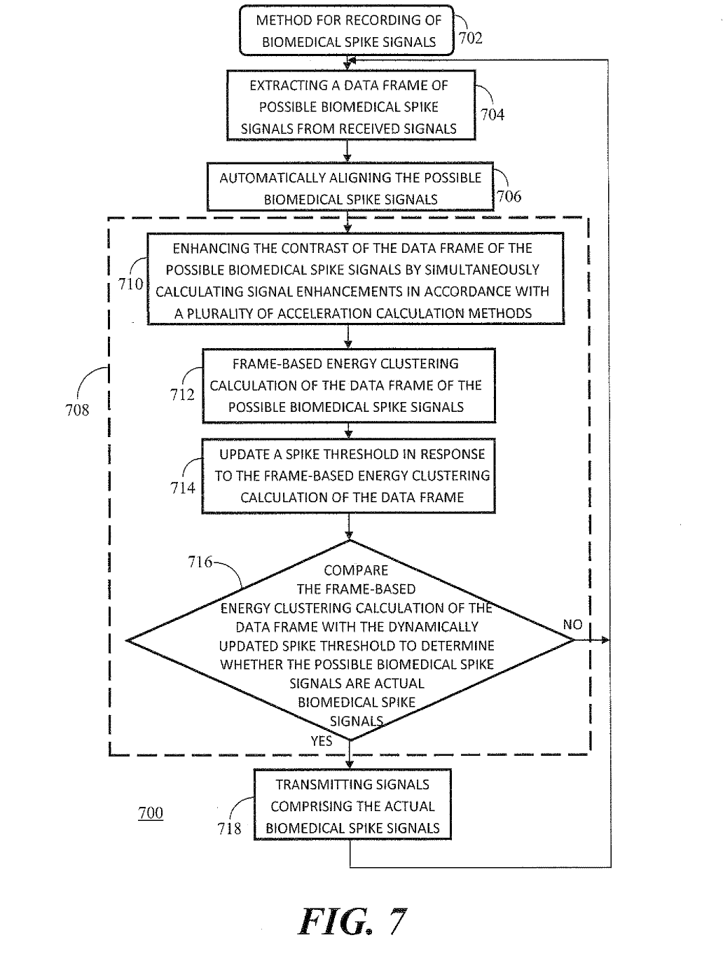

Referring to FIG. 7, a flowchart 700 a method for recording of biomedical spike signals 702 in accordance with the present embodiment. The method initially extracts 704 a data frame of possible biomedical spike signals from received signals. Next, the method automatically aligns 706 the possible biomedical spike signals. Following alignment 706 of the possible biomedical spike signals, the method perform spike detection 708 by determining whether the possible biomedical spike signals are actual biomedical spike signals. The spike detection process 708 includes enhancing contrast 710 by simultaneously calculating signal enhancements in accordance with a plurality of acceleration calculation methods and frame-based energy clustering 712 of the frame-based sample of the possible biomedical spike signals. The spike detection process 708 further includes updating 714 a spike threshold in response to the energy clustering calculation of the frame-based sample of the possible biomedical spike signals and comparing 716 the frame-based energy clustering calculation of the data frame with the dynamically update spike threshold to determine whether the possible biomedical spike signals are actual biomedical spike signals.

If the comparison step 716 determines that the possible spike signals are not actual biomedical spike signals than processing returns to examine additional data frames 704. Only when the comparison step 716 determines that the possible spike signals are actual biomedical spike signals does processing transmit 718 the actual spike signals, thereby significantly reducing energy consumption. After transmission 718, processing returns to examine additional data frames 704.

Thus, it can be seen that the present embodiment can provide a high accuracy method and system for biomedical spike detection which is simultaneously robust against noise and DC drifting. By performing preliminary spike alignment as the first step of data reduction in accordance with the present embodiment, only those frames satisfying specified criteria are sent for energy clustering calculations thereby reducing energy consumption. Preliminary spike alignment also ensures all potential spikes are measured using the energy clustering calculator 234 thereby reducing the missed spike detection rate. Also, the energy clustering index of aligned spikes can be reused in later spike sorting engines without additional hardware cost. While exemplary embodiments have been presented in the foregoing detailed description of the invention, it should be appreciated that a vast number of variations exist. For example, the methods and systems in accordance with the present embodiment can be used for multi-sensor data fusion utilizing spike detection and extracellular EEG recording in addition to implantable wireless neural recording.

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

1. A method for recording biomedical spike signals, wherein the biomedical signals comprise one or more of electrocardiography (ECG) signals, electroencephalography (EEG) signals or neural signals, the method comprising:
extracting possible biomedical spike signals from received signals, wherein the possible biomedical spike signals are serially received signals and wherein a frame-based sample comprises a portion of the serially received signals including a data peak signal and a plurality of signals received before the data peak signal and a plurality of signals received after the data peak signal;
aligning the possible biomedical spike signals in accordance with the data peak signal of each frame-based sample; and
thereafter performing spike detection on the frame-based samples of the possible biomedical spike signals by determining whether the possible biomedical spike signals are actual biomedical spike signals in response to energy clustering of the frame-based sample of the possible biomedical spike signals.

2. The method in accordance with claim 1 wherein the aligning step comprises automatically aligning the possible biomedical spike signals as they are extracted from the received signals.

3. (canceled)

4. (canceled)

5. The method in accordance with claim 1, wherein the step of determining whether the frame-based sample of the possible biomedical spike signals are actual biomedical spike signals comprises determining whether the frame-based sample of the possible biomedical spike signals are actual biomedical spike signals in response to frame-based energy clustering calculations of the frame-based sample of the possible biomedical spike signals.

6. The method in accordance with claim 5 wherein the step of determining whether the frame-based sample of the possible biomedical spike signals are actual biomedical spike signals further comprises comparison of the frame-based energy clustering calculation of the frame-based sample of the possible biomedical spike signals with a spike threshold dynamically updated in response to the energy clustering calculation of each frame-based sample of the possible biomedical spike signals.

7. The method in accordance with claim 5 wherein the step of determining whether the frame-based sample of the possible biomedical spike signals are actual biomedical spike signals further comprises enhancing the frame-based energy clustering calculations of each possible biomedical spike signal by enhancing contrast of each frame-based sample of the possible biomedical spike signal before the energy clustering calculations by simultaneously calculating signal enhancements in accordance with a plurality of acceleration calculation methods.

8. The method in accordance with claim 7 wherein the plurality of acceleration calculation methods comprise one or more acceleration calculation methods selected from the group comprising a first acceleration calculation method for calculating the sum of the absolute value of all multiple data samples within the frame-based sample of the possible biomedical spike signal, a second acceleration calculation method for calculating the squared root of the sum of squared values of each data sample in the multiple data samples within the frame-based sample of the possible biomedical spike signal, and a third acceleration calculation method for calculating the variance of each data sample in the multiple data samples within the frame-based sample of the possible biomedical spike signal.

9. The method in accordance with claim 1 further comprising transmitting signals comprising the actual biomedical spike signals.

10. A biomedical signal recording device for recording biomedical signals selected from electrocardiography (ECG) signals, electroencephalography (EEG) signals and neural signals, the biomedical signal recording device comprising:
a preliminary alignment module for extracting possible biomedical spike signals from received signals while automatically aligning the possible biomedical spike signals, the preliminary alignment module comprising:
an analog-to-digital converter (ADC) for converting serially received analog biomedical signals into serially received discrete-time data signals; and
a memory device coupled to the ADC for capturing a frame of multiple data samples as each possible biomedical spike signal,
wherein each frame of multiple data samples of the serially received discrete-time data signals comprises a data peak signal and a plurality of signals received before the data peak signal and a plurality of signals received after the data peak signal, and wherein the preliminary alignment module automatically aligns the possible biomedical spike signals by frame-based automatically aligning each possible biomedical spike signal in accordance with the data peak signal of each frame-based sample; and
a spike detection module coupled to the preliminary alignment module for receiving the possible biomedical spike signals therefrom and for determining whether the possible biomedical spike signals are actual biomedical spike signals, the spike detection module comprising an energy clustering calculator coupled to the memory of the preliminary alignment module for energy clustering of the possible biomedical spike signals to extract a single valued feature from the multiple data samples of the serially received discrete-time data signals to determine whether the possible biomedical spike signals are actual biomedical spike signals.

11.-13. (canceled)

14. The biomedical recording device in accordance with claim 13 wherein the spike detection module comprises a dynamic threshold module coupled to the energy clustering calculator, the dynamic threshold module comprising:
a dynamic threshold updater for dynamically updating a spike threshold in response to the extracted single valued feature; and
a comparator for comparison of the extracted single valued feature with the dynamically updated spike threshold to determine whether the extracted single valued feature comprises an actual biomedical spike signal.

15. The biomedical recording device in accordance with claim 13 wherein the spike detection module further comprises a plurality of accelerators coupled between the memory device of the preliminary alignment module and the energy clustering calculator for enhancing the frame-based energy clustering calculations of each possible biomedical spike signal by the energy clustering calculator by enhancing contrast of the multiple data samples of the possible biomedical spike signal.

16. The biomedical recording device in accordance with claim 15 wherein the plurality of accelerators comprise one or more accelerators selected from the group comprising a first calculator for calculating the sum of the absolute value of all the multiple data samples within the memory device, a second calculator for calculating the squared root of the sum of squared values of each data sample in the multiple data samples within the memory device, and a third calculator for calculating the variance of each data sample in the multiple data samples within the memory device.

17. The biomedical recording device in accordance with claim 16 wherein the plurality of accelerators comprises the third calculator, and wherein the energy clustering calculator extracts the single valued feature from the multiple data samples in accordance with
$$EC ( {x}_{L} ) = a \times var ( {x}_{L} ) + ( 1 - a ) \frac{{‖ {x}_{L} ‖}_{2}}{{‖ {x}_{L} ‖}_{1} + eps} \qquad ( 1 )$$
where EC(x_{L}) denotes the single valued feature extracted from the multiple data samples of the serially received discrete-time data signals, x_{L} denotes the preliminary biomedical spike, var{•} denotes a variance calculation by the third calculator, eps denotes a constant to prevent zero division, and α denotes a weighting factor selected from a constant weighting factor and a time-varying variable weighting factor.

18. The biomedical recording device in accordance with claim 10 further comprising transmission circuitry for transmitting wireless signals comprising the actual biomedical spike signals, the transmission circuitry including a transmission switch, a transmission module and an antenna, wherein the transmission switch operates under control of the spike detection module for forwarding the actual biomedical spike signals to the transmission module for wireless transmission thereof.

19. (canceled)

20. The biomedical recording device in accordance with claim 19 wherein the biomedical spike signals comprise neural signals, the biomedical recording device further comprising a biocompatible housing for enclosing the preliminary alignment module and the spike detection module for implantable wireless neural recording.

21. The biomedical recording device in accordance with claim 10, wherein the wherein the preliminary alignment module comprises a memory address control unit coupled to the memory device for frame-based automatically aligning each possible biomedical spike signal by aligning each of the multiple data samples captured in the memory device in accordance with the data peak signal of the frame-based sample stored in the memory device.
